(19) United States
(12) Patent Application Publication
(10) Pub. No.: US 2020/0029839 A1
(43) Pub. Date: Jan. 30, 2020

(54) SMART PERSONAL PORTABLE BLOOD PRESSURE MEASURING SYSTEM AND METHOD FOR CALIBRATING BLOOD PRESSURE MEASUREMENT USING THE SAME

(71) Applicant: K-Jump Health Co., Ltd., New Taipei City (TW)

(72) Inventor: CHAO-MAN TSENG, New Taipei city (TW)

(21) Appl. No.: 16/244,475

(22) Filed: Jan. 10, 2019

(30) Foreign Application Priority Data
Jul. 27, 2018 (TW) 107125953

(51) Int. Cl. A61B 5/021; A61B 5/0215; A61B 5/022; A61B 5/024

(52) U.S. Cl. CPC A61B 5/02141; A61B 5/02156; A61B 5/02208; A61B 5/0006; A61B 5/02416; A61B 5/02108; A61B 5/02241; A61B 5/02438

(57) ABSTRACT

A smart personal portable blood pressure measuring system comprises a smart blood pressure measuring base, and a portable blood pressure measuring apparatus comprising a metal electrode detecting unit for detecting an electrocardiography (EKG) signal, a photoplethysmography detector for detecting a photoplethysmography (PPG) signal, a storage unit, a first central processor, a first power supply, and a first coupling interface unit. The storage unit stores a plurality of blood pressure values and a blood pressure algorithm. The first central processor performs a calculation according to the EKG signal, the PPG signal and the blood pressure algorithm. The first power supply provides the necessary power for operating the portable blood pressure measuring apparatus. The first coupling interface unit is coupled to the smart blood pressure measuring base so that the smart blood pressure measuring base is capable of transmitting data to the portable blood pressure measuring apparatus.

### RELATED APPLICATIONS

This application claims the benefit of Taiwan Patent Application Serial No. 107125953, filed Jul. 27, 2018, the subject matter of which is incorporated herein by reference.

### BACKGROUND OF INVENTION

### 1. Field of the Invention

This invention relates to blood pressure measurement, and in particular, it relates to a portable blood pressure measuring system for measuring the blood pressure according to detected electrocardiography (EKG) signal, photoplethysmography (PPG) signal, and a blood pressure algorithm and a method for calibrating the blood pressure algorithm according to cuff-measured blood pressures, and the detected EKG signal and PPG signal.

### 2. Description of the Prior Art

Conventionally, the way for measuring the blood pressure is divided into two different type measurements, including invasive blood pressure measurement as well as non-invasive blood pressure measurement. The invasive blood pressure measurement is commonly used by connecting one end of conducting tubing to a sensor, after exhausting gas and relatively resetting to zero reference point, inserting an arterial line coupled to the conducting tubing into the artery of the examinee, and finally converting the voltage signal detected by the sensor into blood pressure information.

On the other hand, the non-invasive blood pressure measurement can be operated in different kind of ways. The main method of the non-invasive blood pressure measurement is operated by inflating the cuff wrapped around upper arm of the examinee for stopping blood flow through the artery, then deflating the cuff so that the pressure will be gradually reduced whereby the amplitude of the arterial pulses can be detected by pressure sensor. The stronger the arterial pulse is, the larger the amplitude will be. When the amplitude reaches the largest value, the corresponding pressure is regarded as the average arterial pulse pressure. After that, with the detected amplitude is getting smaller and smaller, the cuff pressure will also be reduced. Finally, the amplitude of arterial pulse will not be detected until the cuff pressure is smaller than the diastolic pressure. Please refer to FIG. 1A, which schematically illustrates a principle of blood pressure detection by conventional cuff operation. In FIG. 1A, a pulse amplitude variation versus cuff pressure variation is illustrated. The systolic pressure is determined by allocating a first position having half-maximum amplitude on the pulse amplitude signal curve, and taking the cuff pressure corresponding to the first position as the systolic pressure. The diastolic pressure is determined by backward allocating a second position having amplitude that is 80 percent of the maximum amplitude, and taking the cuff pressure corresponding to the second position as the diastolic pressure. In addition, please refer to FIG. 1B, wherein alternatively, the systolic pressure and diastolic pressure can also be determined according to the known art by determining first and last Korotkoffs sound signal.

However, the inflation/deflation cuff in conventional blood pressure measurement will induce uncomfortable feeling and will also take time during the inflating or deflating operation. In addition, the bulk volume size renders it inconvenient to be carried. In order to improve conventional cuff type measuring device, another non-invasive blood pressure measurement is adopted by using the Electrocardiography (EGG or EKG) and Photopletysmography (PPG) for calculating the blood pressure. For example, a pulse propagating speed calculated by using the EKG and PPG and a compensating pressurizer exerting on the finger or wrist of examinee are both utilized for determining the blood pressure. Nevertheless, the way for obtaining the blood pressure through the combination of EKG and PPG signals is still less accurate. Accordingly, there has a need to improve the accuracy of the non-invasive blood pressure measurement using the EKG and PPG

### SUMMARY OF THE INVENTION

The present invention is directed to a smart personal portable blood pressure measuring system for measuring blood pressure including systolic blood pressure value and diastolic blood pressure value. The system comprises a portable blood pressure measuring apparatus for measuring the blood pressure by utilizing an electrocardiography (EKG) signal, a photoplethysmography (PPG) signal, and a blood pressure algorithm. For improving accuracy of the measurement, diastolic blood pressure values and systolic blood pressure values obtained from cuff measurement of the system are utilized for calibrating the blood pressure algorithm. The calibrated blood pressure algorithm is further stored in the portable blood pressure measuring apparatus so that the user can carry the portable blood pressure measuring apparatus and accurately measure the blood pressure anytime in any environment or occasion.

To achieve the above objects, the present invention provides a portable blood pressure measuring system, which comprises: a smart blood pressure measuring base and a portable blood pressure measuring apparatus releasably coupled to the smart blood pressure measuring base. The portable blood pressure measuring apparatus further comprises a metal electrode detecting unit for detecting an electrocardiography (EKG) signal, a photoplethysmography detector for detecting a photoplethysmography (PPG) signal, a storage unit for storing a plurality of blood pressure values with respect to an examinee/user and storing a blood pressure algorithm, a first central processor configured to perform a calculation according to the EKG signal, the PPG signal and the blood pressure algorithm for obtaining the plurality of blood pressure values, a first power supply configured to provide necessary power for operating the portable blood pressure measuring apparatus, and a first coupling interface unit configured to couple to the smart blood pressure measuring base for transmitting data between the smart blood pressure measuring base and the portable blood pressure measuring apparatus.

In another aspect, the present invention provides a method for calibrating blood pressure measurement, comprising steps of: providing a smart blood pressure measuring base and a portable blood pressure measuring apparatus releasably coupled to the smart blood pressure measuring base, wherein the smart blood pressure measuring base comprises a cuff, the portable blood pressure measuring apparatus comprises a metal electrode detecting unit for detecting an electrocardiography (EKG) signal, and a photoplethysmography detector for detecting a photoplethysmography (PPG) signal; electrically connecting the portable blood pressure measuring apparatus to the smart blood pressure measuring base; measuring a diastolic blood pressure value and a systolic blood pressure value with respect to an examinee through the smart blood pressure measuring base; measuring an electrocardiography (EKG) signal and a photoplethysmography (PPG) signal of the examinee through the portable blood pressure measuring apparatus; obtaining a blood flow value (I) and a blood flow resistance value (R) respectively according to the PPG signal and EKG signal; and using a blood pressure algorithm including a first calculation formula expressed as D1=R×I×fd(x), and a second calculation formula expressed as S1=R×I×fs(x) for calculating the fd(x) and the fs(x), wherein D1 represents a diastolic blood pressure value, S1 represents a systolic blood pressure value, R represents the blood flow resistance value, I represents the blood flow value, fd(x) represents a calibration function of diastolic blood pressure, and fs(x) represents a calibration function of systolic blood pressure.

In another aspect, the present invention provides a portable blood pressure measuring apparatus, comprising a metal electrode detecting unit for detecting an electrocardiography (EKG) signal, a photoplethysmography detector for detecting a photoplethysmography (PPG) signal, a first central processor is configured to perform a calculation according to the EKG signal, the PPG signal and the blood pressure algorithm, a storage unit for storing a plurality of first blood pressure values with respect to an examinee and a blood pressure algorithm, a first central processor configured to perform a calculation according to the EKG signal, the PPG signal and the blood pressure algorithm for obtaining the plurality of first blood pressure values, a first power supply configured to provide necessary power for operating the portable blood pressure measuring apparatus, and a first coupling interface unit configured to couple to a smart blood pressure measuring base for transmitting data between the smart blood pressure measuring base and the portable blood pressure measuring apparatus, wherein the data including a plurality of second blood pressure values for calibrating and modifying the blood pressure algorithm.

It is to be understood that both the foregoing general description and the following detailed descriptions are exemplary and explanatory and are intended to provide further explanation of the invention as claimed.

### BRIEF DESCRIPTION OF THE DRAWINGS

The present invention will now be specified with reference to its preferred embodiment illustrated in the drawings, in which:

FIGS. 1A and 1B schematically illustrate a principle of blood pressure detection in utilizing cuff of the prior art;

FIG. 2 illustrates a portable blood pressure measuring system according to a first embodiment of the present invention;

FIGS. 3A and 3B are respectively illustrate block diagram of a portable blood pressure measuring apparatus, as well as a smart blood pressure measuring base according to one embodiment of the present invention;

FIG. 4 illustrates one embodiment of a photoplethysmography detector for detecting a photoplethysmography (PPG) signal of the present invention;

FIGS. 4A and 4B respectively illustrate different location of finger-engaged area of the portable blood pressure measuring apparatus according to different embodiments of the present invention;

FIG. 4C illustrates configuration of an electrode of the metal electrode detecting unit according to another embodiment of the present invention;

FIGS. 5A and 5B respectively illustrate a portable blood pressure measuring system according to a second and a third embodiment of the present invention;

FIG. 6 schematically illustrates a method for calibrating blood pressure measurement according to one embodiment of the present invention;

FIG. 6A schematically illustrates one embodiment for simultaneously measuring systolic and diastolic blood pressure by blood pressure cuff and acquiring an electrocardiography (EKG) signal and photoplethysmography (PPG) signal;

FIG. 6B schematically illustrates a method for calibrating blood pressure measurement according to a second embodiment of the present invention;

FIG. 7A schematically illustrates a waveform of the electrocardiography (EKG) signal;

FIG. 7B schematically illustrates a waveform of the photoplethysmography (PPG) signal;

FIG. 8 schematically illustrates a waveform combining with the electrocardiography (EKG) signal and the photoplethysmography (PPG) signal within a specific time interval according to one embodiment of the present invention; and

FIG. 9 partially illustrates a part of the photoplethysmography (PPG) signal shown in FIG. 8.

### DESCRIPTION OF THE PREFERRED EMBODIMENT

The invention disclosed herein is directed to a portable blood pressure measuring system and a method for calibrating the blood pressure measurement. In the following description, numerous details corresponding to the aforesaid drawings are set forth in order to provide a thorough understanding of the present invention so that the present invention can be appreciated by one skilled in the art, wherein like numerals refer to the same or the like parts throughout.

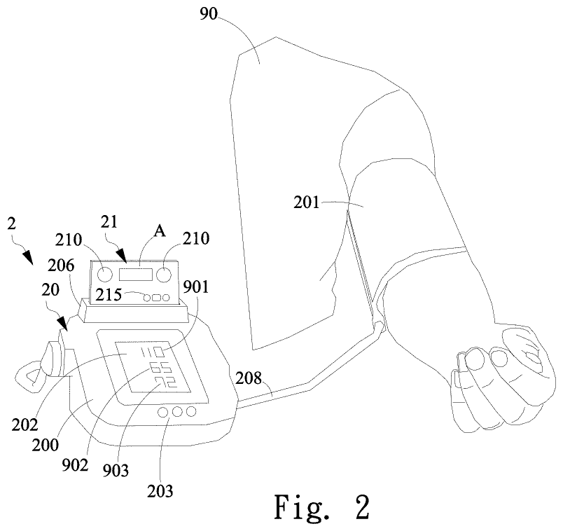

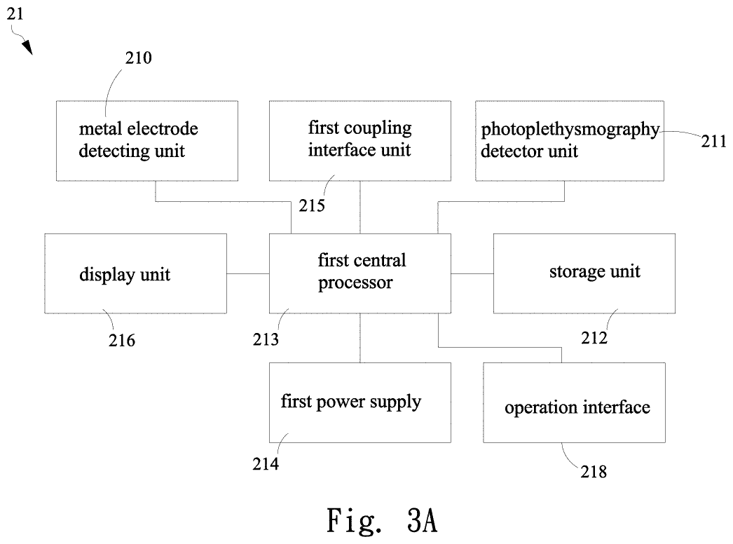

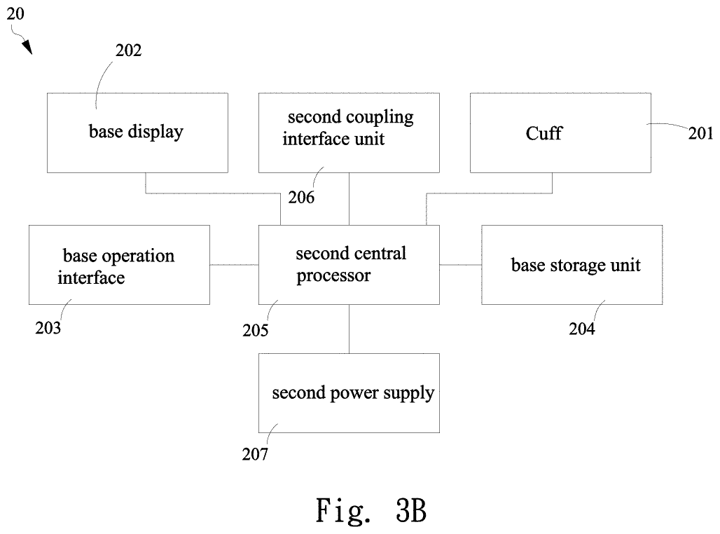

Please refer to FIGS. 2, 3A and 3B, which schematically illustrate a portable blood pressure measuring system and block diagrams thereof according to one embodiment of the present invention, respectively. In the present invention, the system 2 comprises a smart blood pressure measuring base 20 and a portable blood pressure measuring apparatus 21, which is releasably coupled to the smart blood pressure measuring base 20. The portable blood pressure measuring apparatus comprises a metal electrode detecting unit 210 for detecting an electrocardiography (EKG) signal, a photoplethysmography detector unit 211 for detecting a photoplethysmography (PPG) signal, a storage unit 212, a first central processor 213, a first power supply 214, a first coupling interface unit 215, and a display unit 216.

In this embodiment, the metal electrode detecting unit 210 includes at least two electrodes whereby a user or examinee can press onto the two electrodes through fingers of both hands. When the skin of each finger contact the corresponding electrode, the electrodes can detect electrical activity of heart thereby generating electrocardiography with respect to the potential variation of the heart.

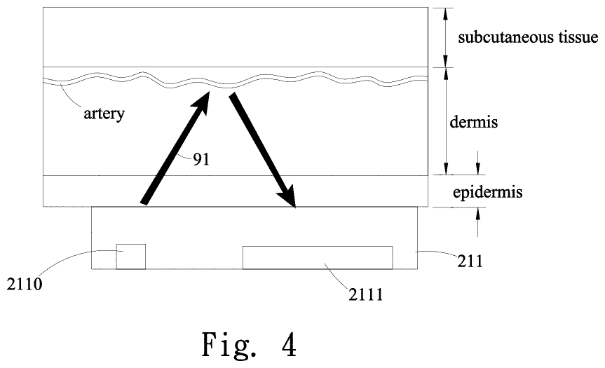

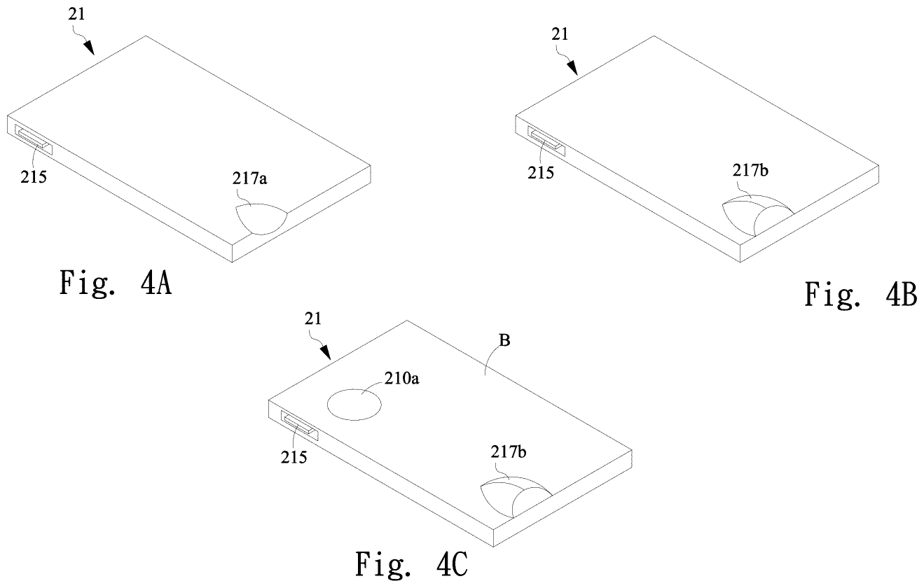

Please refer to FIG. 4, which schematically illustrates the photoplethysmography detector 211 for detecting a photoplethysmography (PPG) signal. In the present embodiment shown in FIG. 4, the detector 211 comprises a light transmitter 2110 and a light receiver 2111. The light transmitter 2110 is utilized to emit least one color of detecting light, for example the red light. It is noted that the light type is not limited to the red light, for example, the infrared light or green light are also known and commercially available to be utilized. In this embodiment, when the detecting light emitted from the light transmitter 2110 and received by the light receiver 2111, the variation of blood flow in the blood vessel can be detected. Please refers to FIGS. 4A and 4B, wherein, in one embodiment, portable blood pressure measuring apparatus 21 comprises a finger-engaged area 217a, or 217b arranged on the a surface opposite to a surface having the metal electrode detecting unit 210. In one embodiment, the finger-engaged area like 217a is formed as a concave structure, and the photoplethysmography detector 211 is set on the concave structure. Alternatively, the finger-engaged area 217b is formed as a tunnel structure where at least one finger of the user can insert therein. Through the configuration of the finger-engaged area 217a or 217b, a light dissipation can be avoided so as to improve accuracy of PPG signal detection.

Additionally, in one embodiment, the portable blood pressure measuring apparatus 21 further comprises an operation interface 218, which is configured to operate the portable blood pressure measuring apparatus 21 and save the measuring data. It is noted that although the photoplethysmography detector 211 and the metal electrode detecting unit 210 are separately arranged at different surfaces, alternatively, the photoplethysmography detector 211 and the metal electrode detecting unit 210 can also be integrated as a multi-function detector arranged at the same surface.

Please refer to FIGS. 2 and 4C. In this embodiment, the portable blood pressure measuring apparatus 21 comprises at least two electrodes, e.g. a pair of electrodes 210 formed on a surface A, whereas the photoplethysmography detector 211, and an electrode 210a are further formed on another surface B opposite to the surface A. The electrode 210a is also configured to detect the electrocardiography (EKG) signal that is utilized to calibrate the EKG signal detected by the two electrodes 210 or is utilized to filter out noise signal of the EKG signal detected by the two electrodes 210.

It is noted that, as illustration in FIGS. 2 and 4C, the user can put thumb fingers of left hand and right hand onto the two electrodes 210 formed on the surface A, respectively, and simultaneously make an index finger of left hand to touch the electrode 210a formed on the surface B, and make index finger of right hand insert into the finger-engaged area 217b or put onto the finger-engaged area 217a having the photoplethysmography detector 211. Alternatively, location of the electrode 210a, and location of the finger-engaged area 217a, or 217b can be also exchangeable according to another embodiments of the present invention.

Please refer back to FIGS. 2, 3A and 3B, wherein the storage unit 212 stores a plurality of blood pressure values with respect to at least one user or examinee, and a blood pressure algorithm. The first central processor 213 performs a calculation according to the EKG signal, PPG signal, and the blood pressure algorithm for obtaining the plurality of blood pressure values. The first power supply 214 is utilized to provide the necessary electrical power to the portable blood pressure measuring apparatus 21. The first coupling interface unit 215 is configured to couple to the smart blood pressure measuring base 20 for allowing data transmitted between the smart blood pressure measuring base 20 and the portable blood pressure measuring apparatus 21. Please refer to FIGS. 4A and 4B, wherein, in one embodiment, the first coupling interface unit 215 is a USB interface. Alternatively, the first coupling interface unit 215 can also be, but should not be limited to, RS232 interface or wireless transmission interface. The display unit 216 is configured to display a plurality of blood pressure values comprising systolic blood pressure values and diastolic blood pressure values obtained by EKG signals, PPG signals, and the blood pressure algorithm. In addition, the display unit 216 can also display non-invasive pulse information calculated from the EKG and PPG signals. The way for calculating the non-invasive pulse information is well-known for the one having ordinary skills in the art, and it will be described hereinafter.

Back to the view of FIGS. 2, 3A and 3B, the smart blood pressure measuring base 20 comprises a base body 200, a cuff 201, a base display 202, a base operation interface 203, a base storage unit 204, a second central processor 205, a second coupling interface unit 206 and a second power supply 207, wherein the cuff 201 is coupled to the base body 200 via an air-conducting tube 208 for obtaining at least one detecting signal from the user. For example, the detecting signal includes sound signal, pressure signal, and etc. The base display 202 is arranged on the base body to display a systolic blood pressure value 901, a diastolic blood pressure value 902 and a non-invasive pulse data 903. The base operation interface 203, in the present embodiment, is arranged on the base body 200, and comprises many physical buttons. Alternatively, the base operation interface 203 can be integrated with the base display 202 for forming a touch-screen base display. Alternatively, in another embodiment, the physical buttons can be a visual type buttons shown on the base display 202 and the visual type buttons are operated through a touch action.

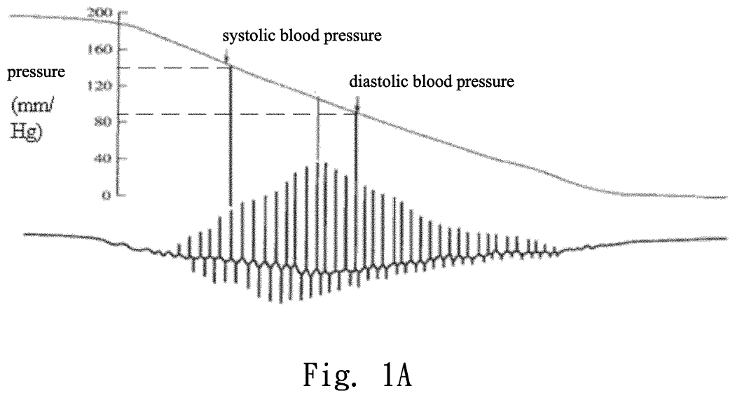

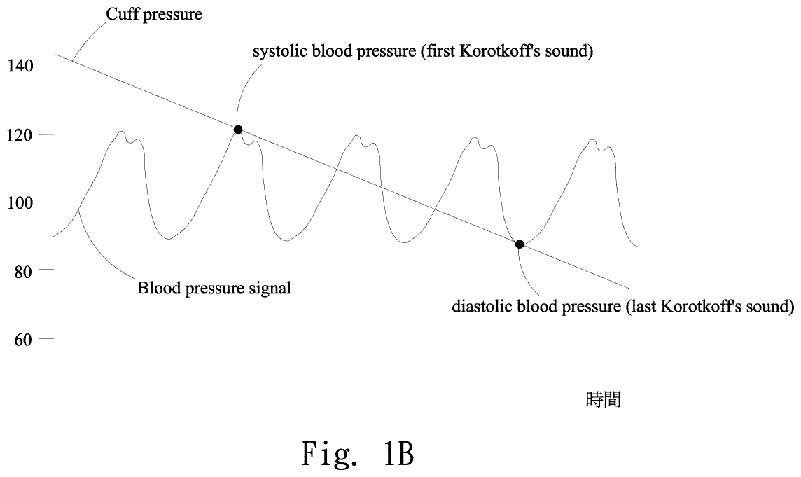

The base storage unit 204 is configured to store the systolic blood pressure value 901, the diastolic blood pressure value 902 and the non-invasive pulse data 903 measured through the cuff 201. The second central processor 205 is configured to perform a calculation according to the detecting signal obtained from the cuff 201 thereby obtaining the systolic blood pressure value 901 and the diastolic blood pressure value 902 according to the well-known art, such as the method shown in FIGS. 1A and 1B, for example. In addition, the second central processor 205 can also determine the non-invasive pulse data 903 according to the detecting signals from the cuff 201. The second coupling interface unit 206 arranged on the base body 200 is coupled to the first coupling interface unit 215 so that the base body 200 is electrically coupled to the portable blood pressure measuring apparatus 21 whereby the data such as the systolic blood pressure value 901, the diastolic blood pressure value 902 and the non-invasive pulse data 903, for example, can be transmitted between the portable blood pressure measuring apparatus 21 and smart blood pressure measuring base 20. In one embodiment, the systolic blood pressure value 901, and the diastolic blood pressure value 902 are transmitted to the portable blood pressure measuring apparatus 21 so as to calibrate the blood pressure algorithm. In one embodiment, the second coupling interface unit 206 is a USB interface. Alternatively, the second coupling interface unit 206 can also be, but should not be limited to, RS232 interface or wireless transmission interface. The second power supply 207 is configured to provide the necessary power for operating the smart blood pressure measuring base 20.

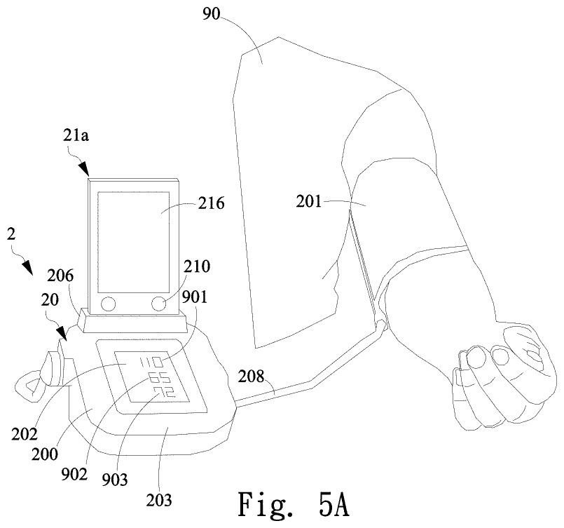

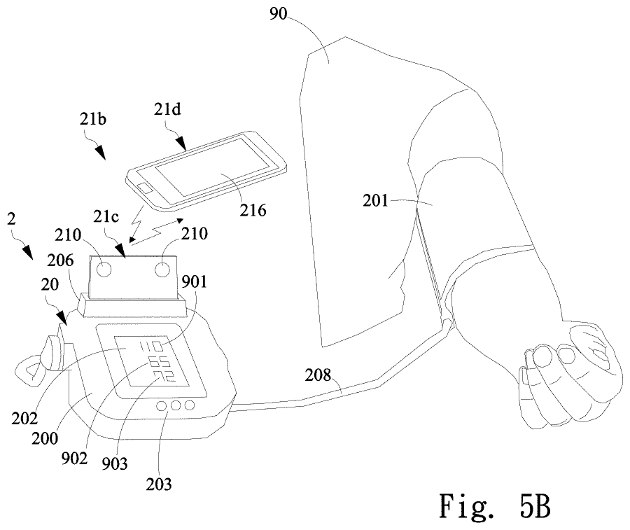

Moreover, in the embodiment shown in FIG. 2, the portable blood pressure measuring apparatus 21 is formed as a card structure. In addition to the card structure, for example, in another embodiment shown as FIG. 5A, a portable blood pressure measuring apparatus 21a is an electronic communication device such as smart phone or tablet, for example. The electronic communication device comprises the photoplethysmography detector unit, e.g. arranged on the back surface of the portable blood pressure measuring apparatus 21a, for detecting the PPG signal, and the metal electrode detecting unit 210 for detecting the EKG signal. It is noted that although the portable blood pressure measuring apparatus 21a is electrically coupled to the smart blood pressure measuring base 20 through a wire connection, in another embodiment, the portable blood pressure measuring apparatus 21a can be wirelessly coupled to the smart blood pressure measuring base 20 for transmitting the data. In the present embodiment, the portable blood pressure measuring apparatus 21a obtains the EKG signal and the PPG signal via an application (APP). The APP is also utilized to calculate the systolic blood pressure value, the diastolic blood pressure value and the non-invasive pulse data according to the detected EKG signal, PPG signal and the blood pressure algorithm. The systolic blood pressure value, the diastolic blood pressure value and the non-invasive pulse data are shown on the display unit 216. The display unit 216 can be utilized to display the operation interface after executing the APP, whereby the user can operate the blood pressure measurement and access the measured data.

Please refer to FIG. 5B. In this embodiment, a portable blood pressure measuring apparatus 21b comprises a card structure 21c and an electronic communication device 21d, wherein the card structure 21c has the photoplethysmography detector unit for detecting the PPG signal, and the metal electrode detecting unit 210 for detecting the EKG signal, and the first central processor 213 and the display unit 216 are separately arranged on the card structure 21c and electronic communication device 21d. The electronic communication device 21d can communicate with the card structure 21c through cable communication or wireless communication.

In the above-mentioned multiple smart personal portable blood pressure measuring system 2, since the portable blood pressure measuring apparatus 21 and the smart blood pressure measuring base 20 are separately arranged, the user can carry the portable blood pressure measuring apparatus 21 and measure the blood pressure, heartbeat, or pulse status anytime and anywhere through the portable blood pressure measuring apparatus 21 for managing and monitoring the healthy status of the user immediately. However, since the blood pressure will be varied with the age, body shape, life environment, or living habit, in order to accurately measure the blood pressure without the influence of above-mentioned conditions, the blood pressure algorithm stored in the portable blood pressure measuring apparatus 21 can be calibrated and updated through the blood pressures, heartbeat and pulse measured by the cuff 201 coupled to the smart blood pressure measuring base 20 whereby the user can accurately measure the blood pressure, heartbeat or pulse through the portable blood pressure measuring apparatus 21 anytime and anywhere. Accordingly, not only can the smart personal portable blood pressure measuring system 2 solve the inaccurate problem of blood pressure measurement obtained from the EKG and PPG signals, but also the operation convenience for measuring blood pressure immediately can be provided.

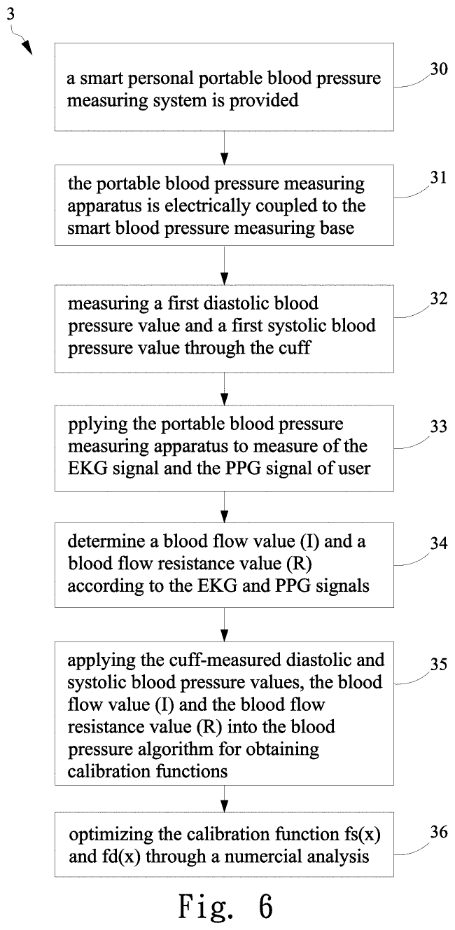

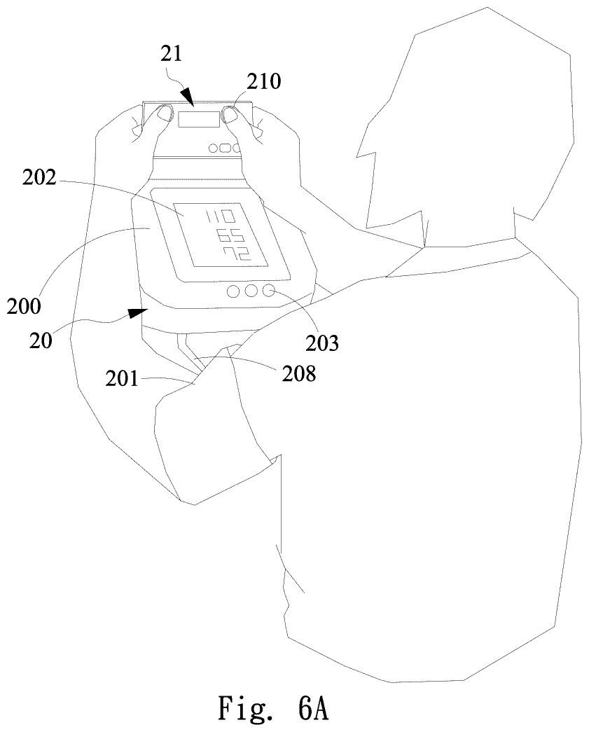

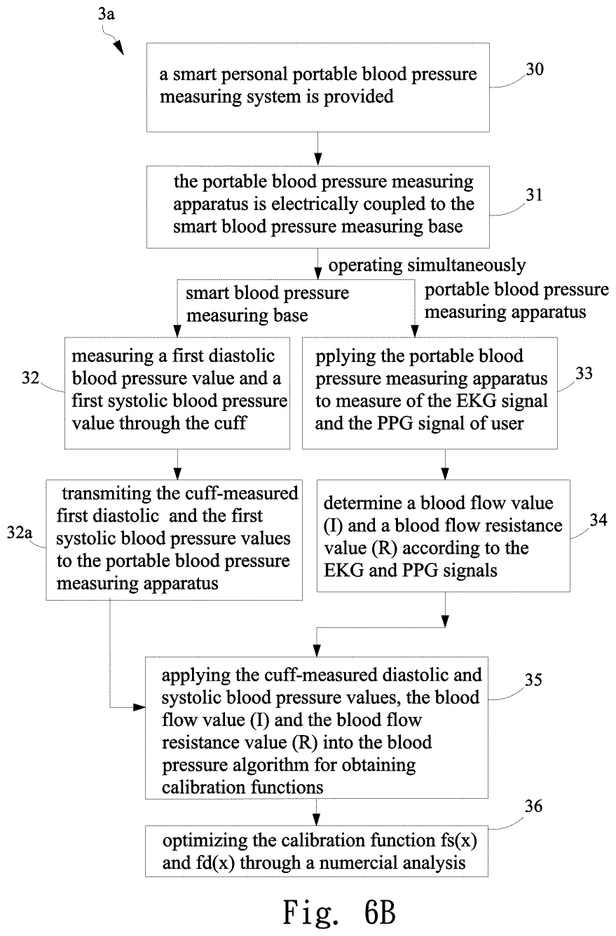

Please refer to FIG. 6, which illustrates schematically a flow chart of the method for calibrating blood pressure measurement using the smart personal portable blood pressure measuring system. The steps of method 3 are explained blow. In the step 30, a smart personal portable blood pressure measuring system comprises a smart blood pressure measuring base and a portable blood pressure measuring apparatus releasably coupled to the smart blood pressure measuring base is provided. In one embodiment, the portable blood pressure measuring system can be one of the embodiments shown in FIGS. 2, 5A and 5B. In the following explanation, the system shown in FIG. 2 is utilized for explaining the steps of the method 3. In the step 31, the portable blood pressure measuring apparatus 21 is electrically coupled to the smart blood pressure measuring base 20. In the present embodiment, the smart blood pressure measuring base 20 comprises the second coupling interface unit 206 structured as a slot having an electrical interface formed inside the slot, wherein the electrical interface is corresponding to a specific communication protocol, such as USB or RS232, for example. The portable blood pressure measuring apparatus 21 is inserted into the slot whereby the first coupling interface unit 215 is electrically coupled to the second coupling interface unit 206.

Next, the step 32 is performed by measuring a first diastolic blood pressure value and a first systolic blood pressure value through cuff 201 of the smart blood pressure measuring base 20. In the present step, the cuff 201 is utilized to wrap around upper arm of the user for measuring the blood pressure. In order to accurately calibrate the blood pressure algorithm, it is necessary to use the accurate blood pressure information as calibrating parameter. Since the blood pressure measured by the cuff 201 will be more accurate, the cuff-measured blood pressure values can be utilized as the calibrating parameter for calibrating the blood pressure algorithm. In one embodiment, a plurality of the cuff-measured blood pressures can be obtained and are stored in the storage unit in the smart blood pressure measuring base 20.

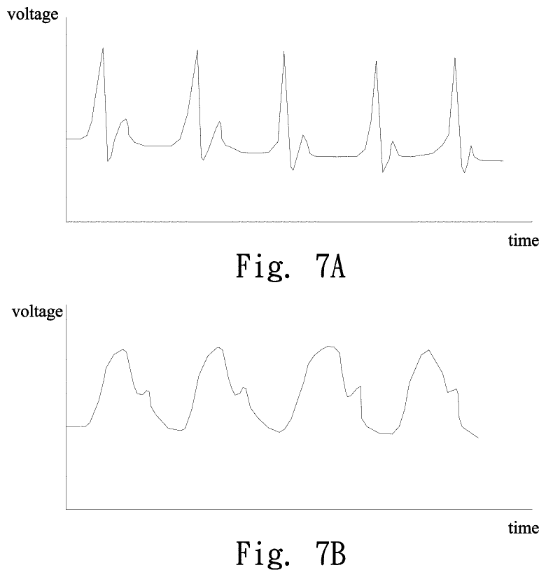

After the step 32, the step 33 is performed by applying the portable blood pressure measuring apparatus 21 to measure of the EKG signal and the PPG signal of the user. In the one embodiment of the present step, the electrode detecting unit 210 and the photoplethysmography detector unit 211 are respectively utilized to measure the EKG and PPG signals. Please refer to FIGS. 7A and 7B, which illustrates the EKG signal and PPG signal, respectively. Through the step 33, parameters for determining a non-invasive blood pressure without using the cuff can be obtained.

After the step 33, the step 34 is operated to determine a blood flow value (I) and a blood flow resistance value (R) according to the EKG and PPG signals. The PPG signal refers to a variation of the blood volume in the blood vessel. The PPG signal is generated according to optical energy absorbs by the optical sensing element wherein the absorbed optical energy represents the variation of optical light caused by the blood flow and pulse of the blood vessel. Since the blood flow rate, i.e. flow volume with respect to the cross-sectional area, will be varied corresponding to the heartbeats, the sensing potential generated by the optical sensing element will also be varied with respect to the blood volume. It is noted that the timing that the most part of the optical light is absorbed represents a systole of the heat; therefore, the amplitude of the PPG signal will be proportional to blood volume flowing into or out from the heat. When an optical light having a specific optical wavelength is projected onto the finger, the intensity of the reflected or penetrated optical light absorbed by the optical sensing element will reflect the optical absorption of the blood in the blood vessel of the projected finger. Accordingly, the PPG signal can represents blood volume from the heat to the projected finger during a cycle of systole and diastole of the heat, wherein the blood volume can be associated with the blood flow value (I) and the blood flow resistance value (R).

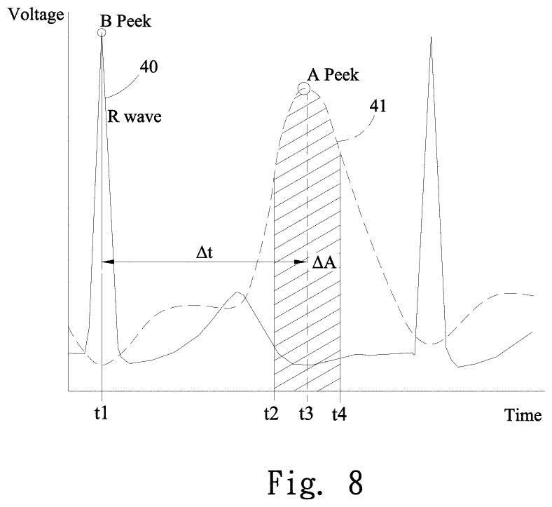

On the other hand, since the EKG signal represents a tiny potential variation on the skin, which is induced by each heartbeat of the heart. After amplifying the tiny potential variation, the waveform of the electrocardiography is shown as FIG. 7A. Please refer to FIG. 8. In the embodiment, the PPG signal 41 is a detected result corresponding to the blood flowing to the fingertip. Thus, the time point when the PPG signal 41 is detected is slower than a time point when the EKG signal 40 is measured; therefore, a time interval (Δt) is generated between the EKG signal 40 and PPG signal 41. The time interval (Δt) is defined between a first characteristic point A of the PPG signal 41 and a second characteristic point B of the EKG signal 40 relevant to the PPG signal 41, in which the first characteristic point A of the PPG signal 41 is defined as a point having maximum slope on the main wave crest of the PPG signal 41 at a first time point (t1) while the second characteristic point B represents a peak point of R wave of the EKG signal 40 corresponding to the PPG signal 41 at a second time point (t3).

According to the time interval (Δt) described above, it is capable of determining the blood flow resistance value (R) and blood flow value (I). In one embodiment, the blood flow resistance value (R) can be defined as the time interval (Δt) multiplied by a function value (k1), i.e. R=Δt×k1(Δt), wherein k1(Δt) is a constant value or a function varied with interval (Δt), which can be determined by the user and can be adjusted according to numerical analysis among the cuff-measured blood pressure values. The blood flow value (I) is equal to an integral value (ΔA) with respect to a specific curve segment of the PPG signal multiplied by a function value (k2), i.e. I=ΔA×k2(ΔA), wherein and the function value (k2) is varied with the integral value (ΔA) or a constant value, which can be determined by the user and the function value (k2) can be adjusted according to numerical analysis among the cuff-measured blood pressure values. It is noted that the specific curve segment of PPG signal is up to the user's choice. For example, the specific curve segment can be a segment between t2˜t4 shown in FIG. 8.

After determining the blood flow value (I) and the blood flow resistance value (R), a step 35 is performed by applying the cuff-measured diastolic blood pressure value, a cuff-measured systolic blood pressure value, the blood flow value (I) and the blood flow resistance value (R) into the blood pressure algorithm for obtaining calibration functions. In one embodiment, the blood pressure algorithm includes a first calculation formula expressed as D1=R×I×fd(x), and a second calculation formula expressed as S1=R×I×fs(x), wherein D1 represents a diastolic blood pressure value, S1 represents a systolic blood pressure value, R represents a blood flow resistance value, I represents a blood flow value, fd(x) represents a calibration function of diastolic blood pressure, and fs(x) represents a calibration function of systolic blood pressure. In one embodiment, the calibration algorithm can be performed at the smart blood pressure measuring base 20 by transmitting the blood flow resistance value (R) and the blood flow value (I) to the smart blood pressure measuring base 20. Alternatively, the calibration algorithm can be performed at the portable blood pressure measuring apparatus 21 by transmitting the cuff-measured diastolic and systolic blood pressure values to the portable blood pressure measuring apparatus 21. Alternatively, the calibration algorithm can be performed at a cloud sever by transmitting the cuff-measured diastolic blood pressure value, a cuff-measured systolic blood pressure value, the blood flow value (I), the blood flow resistance value (R) and the blood pressure algorithm to the cloud server. In the following, exemplary embodiments are provided to explain the step 35 for determining fs(x) and fd(x) in detail.

Please refer to FIG. 6A. In one embodiment, in case of the fs(x) and fd(x) are constant values, when the step 32 and step 33 are both executed by the user for obtaining cuff-measured diastolic and systolic blood pressure as well as EKG and PPG signals at the same time. The corresponding detected EKG and PPG signals are shown in FIG. 8. The formula (1) for calculating systolic blood pressure and the formula (2) for calculating diastolic blood pressure are listed as below:

S1=[Δt×k1(Δt)]×[ΔA×k2(ΔA)]×fs(x) (1)

D1=[Δt×k1(Δt)]×[ΔA×k2(ΔA)]×fd(x) (2),

wherein Δt×k1 (Δt) represents the blood flow resistance value (R) and ΔA×k2 (ΔA) represents the blood flow value (I).

As above, it is assume that k1(Δt) and k2(ΔA) are constant value determined by user, which may be the same or different. Although fs(x) and fd(x) is unknown, S1 and D1 is determined as the known cuff-measured systolic and diastolic blood pressure, and [Δt×k1 (Δt)]×[ΔA×k2 (ΔA)] can be determined according the relationship between the PPG and EKG signals shown in FIG. 8. Thus the fs(x) and fd(x) can be capable of being determined from the formula (1) and the formula (2).

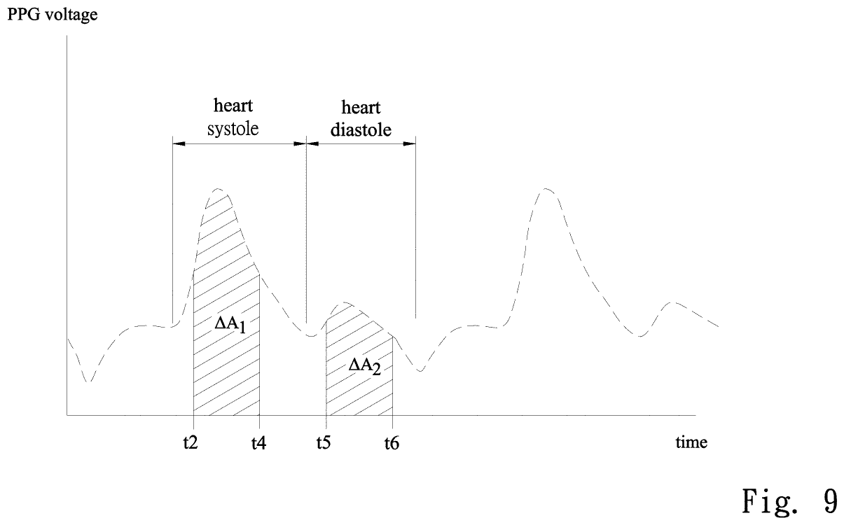

In addition, in the other embodiment shown in FIG. 9. In this embodiment, the ΔA of the formula (1) and the formula (2) is not the same as each other. According to the characteristic of PPG signal, the PPG signal can be divided into two parts respectively corresponding to the diastolic blood pressure and systolic blood pressure. Therefore, an integral value (ΔA1) shown in FIG. 9 is define as the ΔA of the formula (1), while an integral value (ΔA2) shown in FIG. 9 is define as the ΔA of the formula (2). Through the two different integral vales (ΔA1) and (ΔA2), the fs(x) and fd(x) can be calculated as well.

Moreover, in another embodiment that the fs(x) and fd(x) are not the constant value, assuming that the fs(x) is the function of Δt and ΔA1 shown in FIG. 9, and fd(x) is the function of Δt and ΔA2 shown in FIG. 9, which are respectively listed as below:

fs(x)=[aΔt+bΔA1] (3)

fd(x)=[aΔt+bΔA2] (4)

As the formulas shown above, coefficient “a” and “b” of formula (3) and formula (4) can be determined through the blood pressure algorithm expressed as the formulas (5) and (6) shown below. The formula (5) is expressed by substituting formula (3) into formula (1) while the formula (6) is expressed by substituting formula (4) into formula (2).

S1=[Δt×k1(Δt)]×[ΔA1×k2(ΔA1)]×[aΔt+bΔA1] (5)

D1=[Δt×k1(Δt)]×[ΔA2×k2(ΔA2)]×[aΔt+bΔA2] (6)

In the present embodiment, since the parameters including Δt'ΔA1 and ΔA2 can be known according to the FIG. 9, respectively, and the cuff-measured blood pressure values S1 and D1 are also known, the coefficient “a” and “b” in the formulas (5) and (6) can be solved whereby the calibration function of systolic and diastolic fs(x) and fd(x) can be determined. It is known that although the parameter ΔA in formulas (3) and (4) are ΔA1 and ΔA2 shown in FIG. 9, alternatively, in another embodiment, the parameter ΔA can be the ΔA shown in FIG. 8.

In order to improve the accuracy of the blood pressure calculated through the blood pressure algorithm, a step 36 is further operated to optimize the calibration function fs(x) and fd(x) through a numerical analysis by using a plurality of cuff-measured systolic blood pressure values S1˜Sn and a plurality of cuff-measured diastolic blood pressure values D1˜Dn. In the step 36, the steps 32 to 35 are repeatedly operated a plurality of times for obtaining the plurality of systolic and diastolic blood pressure values S1˜Sn and D1˜Dn as well as the plurality of blood flow values (I) and a blood flow resistance values (R) obtained from the associated EKG and PPG signals respectively corresponding to the plurality of cuff-measured blood pressure values (S1, D1)˜(Sn, Dn). After obtaining the plurality of cuff-measured blood pressure values (S1, D1)˜(Sn, Dn), and the plurality of blood flow values (I) and a blood flow resistance values (R) by repeating steps 32-35 a plurality of times, it is capable of using the formulas (1) and (2) or formulas (5) and (6) for obtaining a plurality of sets of calibration function (fs(x), fd(x)).

Taking the formulas (1) and (2) as an example for explaining the step 36. When a plurality of (S1, D1)˜(Sn, Dn), and the corresponding blood flow values (I) and blood flow resistance values (R) are obtained, it is capable of obtaining the plurality of sets of calibration function (fs(x), fd(x)). After that, the numerical analysis, such as linear regression analysis or ensemble average, for example, is utilized to optimizing the fs(x) and fd(x). Likewise, when the formulas (5) and (6) are utilized, a plurality of coefficient values “a” and coefficient values “b” are obtained, the numerical analysis, such as linear regression analysis or ensemble average, for example, is utilized to optimizing the coefficient value “a” and coefficient value “b”, whereby the calibration function fs(x) and fd(x) can be optimized.

The obtained blood pressure algorithm in step 35 or optimized blood pressure algorithm through step 36 is stored in the storage unit of the portable blood pressure measuring apparatus 21. After the blood pressure algorithm is stored, the portable blood pressure measuring apparatus 21 can be released from the smart blood pressure measuring base 20 and the user can carry the portable blood pressure measuring apparatus 21 for measuring the blood pressure non-invasively anytime and anywhere. With the growth of age of the user, change of body shape, or intentionally calibrating the blood pressure algorithm, the user can perform the steps 30 to 36 for calibrating or optimizing the blood pressure algorithm. Alternatively, the portable blood pressure measuring apparatus 21 can record a plurality of blood pressure algorithms respectively corresponding to different user so that the portable blood pressure measuring apparatus 21 can be utilized by different user.

Please refer to FIG. 6B, which illustrates schematically a flow chart of a method for calibrating blood pressure measurement according to a second embodiment of the present invention. The main difference between the embodiments shown in FIG. 6 and FIG. 6A is that the step 32 and the step 33 are being proceeded simultaneously, wherein the step 32 is performed by applying the smart blood pressure measuring base to measure blood pressure values of the user for obtaining a first diastolic blood pressure value and a first systolic blood pressure value through the cuff thereof, and the step 33 is performed by applying the portable blood pressure measuring apparatus 21 to measure of an electrocardiography (EKG) signal and a photoplethysmography (PPG) signal of the user. After the step 32, a step 32a is performed to transmit the cuff-measured first diastolic blood pressure value and the first systolic blood pressure value to the portable blood pressure measuring apparatus 21 while after the step 33, the step 34 is performed to respectively determine the blood flow value (I) and the blood flow resistance value (R) according to the EKG and PPG signals, which is clearly described in the previous embodiment, and will not be further explained hereinafter. After that, the step 35 is performed to obtaining the calibration function fs(x) and fd(x) by using the first diastolic and systolic blood pressure values, the blood flow value (I) and the blood flow resistance value (R), and the blood pressure algorithm such as formulas (1) and (2) or formulas (5) and (6). Finally, in order to improve the accuracy of the blood pressure algorithm, the step 36 can be performed to optimize the blood pressure algorithm through a numerical analysis on the plurality of sets of (fd(x), fs(x)), which is clearly described in the previous embodiment, and will be further described hereinafter.

It will be apparent to those skilled in the art that various modification and variations can be made without departing from the spirit or scope of the invention. Thus, it is intended that the present invention cover modifications and variations that come within the scope of the appended claims and their equivalents.

While the present invention has been particularly shown and described with reference to a preferred embodiment, it will be understood by those skilled in the art that various changes in form and detail may be without departing from the spirit and scope of the present invention.

What is claimed is:

1. A smart personal portable blood pressure measuring system, comprising:
a smart blood pressure measuring base;
a portable blood pressure measuring apparatus releasably coupled to the smart blood pressure measuring base, comprising:
a metal electrode detecting unit for detecting an electrocardiography (EKG) signal;
a photoplethysmography detector for detecting a photoplethysmography (PPG) signal;
a storage unit for storing a plurality of blood pressure values with respect to an examinee and storing a blood pressure algorithm;
a first central processor configured to perform a calculation according to the EKG signal, the PPG signal and the blood pressure algorithm for obtaining the plurality of blood pressure values;
a first power supply configured to provide necessary power for operating the portable blood pressure measuring apparatus; and
a first coupling interface unit configured to couple to the smart blood pressure measuring base for transmitting data between the smart blood pressure measuring base and the portable blood pressure measuring apparatus.

2. The system of claim 1, wherein the portable blood pressure measuring apparatus is a card structure, comprising an operation interface, a finger-engaged area having the photoplethysmography detector, and a display unit for displaying the plurality of blood pressure values, which comprises a systolic blood pressure value and a diastolic blood pressure value.

3. The system of claim 1, wherein the blood pressure algorithm includes a first calculation formula expressed as D1=R×I×fd(x), and a second calculation formula expressed as S1=R×I×fs(x), wherein D1 represents a diastolic blood pressure value, S1 represents a systolic blood pressure value, R represents a blood flow resistance value, I represents a blood flow value, fd(x) represents a calibration function of diastolic blood pressure, and fs(x) represents a calibration function of systolic blood pressure.

4. The system of claim 3, wherein a time interval (Δt) is defined between a first characteristics point of the PPG signal and a second characteristics point of the EKG signal relevant to the PPG signal, in which the first characteristics point of the PPG signal is peak of the PPG signal at a first time point and the second characteristics point of the EKG signal is peak of the EKG signal at a second time point.

5. The system of claim 3, wherein the blood flow resistance value (R) is equal to the time interval (Δt) multiplied by a function value (k1), and the function value (k1) is a function varied with the time interval (Δt) or the function value (k1) is a constant value.

6. The system of claim 3, wherein the blood flow value (I) is equal to an integral value (ΔA) with respect to a curve of the PPG signal multiplied by a function value (k2), and the function value (k2) is a function varied with the integral value (ΔA) or the function value (k2) is a constant value.

7. A method for calibrating blood pressure measurement, comprising steps of:
providing a smart blood pressure measuring base and a portable blood pressure measuring apparatus releasably coupled to the smart blood pressure measuring base, wherein the smart blood pressure measuring base comprises a cuff, the portable blood pressure measuring apparatus comprises a metal electrode detecting unit for detecting an electrocardiography (EKG) signal, and a photoplethysmography detector for detecting a photoplethysmography (PPG) signal;
electrically connecting the portable blood pressure measuring apparatus to the smart blood pressure measuring base;
measuring a diastolic blood pressure value and a systolic blood pressure value with respect to an examinee through the smart blood pressure measuring base;
measuring an electrocardiography (EKG) signal and a photoplethysmography (PPG) signal of the examinee through the portable blood pressure measuring apparatus;
obtaining a blood flow value (I) and a blood flow resistance value (R) respectively according to the PPG signal and EKG signal; and
using a blood pressure algorithm including a first calculation formula expressed as D1=R×I×fd(x), and a second calculation formula expressed as S1=R×I×fs(x) for calculating the fd(x) and the fs(x), wherein D1 represents a diastolic blood pressure value, S1 represents a systolic blood pressure value, R represents the blood flow resistance value, I represents the blood flow value, fd(x) represents a calibration function of diastolic blood pressure, and fs(x) represents a calibration function of systolic blood pressure.

8. The method of claim 7, wherein a time interval (Δt) is defined between a first characteristics point of the PPG signal and a second characteristics point of the EKG signal relevant to the PPG signal, in which the first characteristics point of the PPG signal is peak of the PPG signal at a first time point and the second characteristics point of the EKG signal is peak of the EKG signal at a second time point.

9. The method of claim 8, wherein the blood flow resistance value (R) is equal to the time interval (Δt) multiplied by a function value (k1), and the function value (k1) is with a function varied with the time interval (Δt) or the function value (k1) is a constant value.

10. The method of claim 8, wherein the blood flow value (I) is equal to an integral value (ΔA) with respect to a curve of the PPG signal multiplied by a function value (k2), and the function value (k2) is varied with the integral value (ΔA) or the function value (k2) is a constant value.

11. The method of claim 7, further comprising steps of measuring a first non-invasive pulse data through the smart blood pressure measuring base, and calculating to obtain an oxygen saturation value and a second non-invasive pulse data according to the PPG signal.

12. The method of claim 7, further comprising a step of obtaining a plurality of the diastolic blood pressure values and the systolic blood pressure values for calibrating fd(x) and fs(x) and optimizing the blood pressure algorithm.

13. A portable blood pressure measuring apparatus, comprising:
a metal electrode detecting unit for detecting an electrocardiography (EKG) signal;
a photoplethysmography detector for detecting a photoplethysmography (PPG) signal;
a storage unit for storing a plurality of first blood pressure values with respect to an examinee and a blood pressure algorithm;
a first central processor configured to perform a calculation according to the EKG signal, the PPG signal and the blood pressure algorithm for obtaining the plurality of first blood pressure values;
a first power supply configured to provide necessary power for operating the portable blood pressure measuring apparatus; and
a first coupling interface unit configured to couple to a smart blood pressure measuring base for transmitting data between the smart blood pressure measuring base and the portable blood pressure measuring apparatus;
wherein the data including a plurality of second blood pressure values from the smart blood pressure measuring base for calibrating and modifying the blood pressure algorithm.

14. The apparatus of claim 13, wherein the portable blood pressure measuring apparatus is a card structure, comprising an operation interface, a finger-engaged area having the photoplethysmography detector, and a display unit configured to display a plurality of the first blood pressure values comprising a systolic blood pressure value and a diastolic blood pressure value.

15. The apparatus of claim 13, wherein the blood pressure algorithm includes a first calculation formula expressed as D1=R×I×fd(x), and a second calculation formula expressed as S1=R×I×fs(x), wherein D1 represents a diastolic blood pressure value, S1 represents a systolic blood pressure value, R represents a blood flow resistance value, I represents a blood flow value, fd(x) represents a calibration function of diastolic blood pressure, and fs(x) represents a calibration function of systolic blood pressure.

16. The apparatus of claim 15, wherein a time interval (Δt) is defined between a first characteristics point of the PPG signal and a second characteristics point of the EKG signal relevant to the PPG signal, in which the first characteristics point of the PPG signal is peak of the PPG signal at a first time point and the second characteristics point of the EKG signal is peak of the EKG signal at a second time point.

17. The apparatus of claim 15, wherein the blood flow resistance value (R) is equal to the time interval (Δt) multiplied by a function value (k1), and the function value (k1) is a function varied with the time interval (Δt) or the function value (k1) is a constant value.

18. The apparatus of claim 15, wherein the blood flow value (I) is equal to an integral value (ΔA) with respect to a curve of the PPG signal multiplied by a function value (k2), and the function value (k2) is a function varied with the integral value (ΔA) or the function value (k2) is a constant value.
